SEQUENCE LISTING (11 sequences)

SEQ ID NO: 1 - protein, 124 aa, synthetic construct
  source 1..124: note = HCVR
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
  source 1..8: note = HCDR1
GFTFRDYA

SEQ ID NO: 4 - protein, 8 aa, synthetic construct
  source 1..8: note = HCDR2
ISGSGGNT

SEQ ID NO: 5 - protein, 18 aa, synthetic construct
  source 1..18: note = HCDR3
AKDRLSITIRPRYYGLDV

SEQ ID NO: 6 - protein, 11 aa, synthetic construct
  source 1..11: note = LCDR1
QSLLYSIGYNY

SEQ ID NO: 7 - skipped

SEQ ID NO: 8 - protein, 9 aa, synthetic construct
  source 1..9: note = LCDR3
MQALQTPYT

SEQ ID NO: 9 - protein, 451 aa, synthetic construct
  source 1..451: note = HC aa1-124: HCVR; aa125-451: HC constant
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
  source 1..219: note = LC aa1-112: LCVR; aa113-219: LC constant
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, synthetic construct
  source 1..207: note = hIL-4Ralpha
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

(19) United States
(12) Patent Application Publication
(10) Pub. No.: US 2024/0082353 A1
(43) Pub. Date: Mar. 14, 2024

(54) METHODS FOR PREVENTING OR TREATING ALLERGY BY ADMINISTERING AN IL-4R ANTAGONIST

(71) Applicants: REGENERON PHARMACEUTICALS, INC., Tarrytown, NY (US); SANOFI BIOTECHNOLOGY, Paris (FR)

(72) Inventors: Jennifer D. HAMILTON, Ridgefield, CT (US); Brian N. SWANSON, Paris (FR)

(21) Appl. No.: 18/452,155

(22) Filed: Aug. 18, 2023

Related U.S. Application Data
(63) Continuation of application No. 16/329,184, filed on Feb. 27, 2019, now Pat. No. 11,771,743.
(60) Provisional application No. 62/382,501, filed on Sep. 1, 2016.
(60) Provisional application No. 62/425,726, filed on Nov. 23, 2016.

(51) Int. Cl. A61K 38/17; A61K 9/00; A61K 39/00; A61P 17/00; A61P 37/08; C07K 16/28; G01N 33/569

(52) U.S. Cl. CPC A61K 38/1774; A61K 9/0019; A61K 39/00; A61P 17/00; A61P 37/08; C07K 16/2866; G01N 33/56966; A61K 2039/505; A61K 2039/545; A61K 45/06; C07K 2317/76

(57) ABSTRACT

The present invention provides methods for preventing or treating allergy. Also provided are methods for reducing the susceptibility to an allergen in a subject in need thereof. In certain embodiments, the subject has a disease or disorder selected from the group consisting of atopic dermatitis, asthma, allergic rhinitis, and eosinophilic esophagitis. The methods of the present invention comprise administering to a subject in need thereof a pharmaceutical composition comprising an interleukin-4 receptor (IL-4R) antagonist such as an anti-IL-4R antibody.

### CROSS-REFERENCE TO RELATED APPLICATIONS

This application is a continuation of U.S. patent application Ser. No. 16/329,184, filed Feb. 27, 2019, which is a U.S. National Stage application of PCT/US2017/049538, filed Aug. 31, 2017, which claims benefit of U.S. Provisional patent application No. 62/382,501, filed Sep. 1, 2016, and U.S. Provisional patent application No. 62/425,726, filed Nov. 23, 2016, which applications are incorporated herein by reference. To the extent appropriate, a claim of priority is made to each of the above-disclosed applications.

### REFERENCE TO A SEQUENCE LISTING XML

The instant application contains a Sequence Listing which has been submitted electronically in XML format and is hereby incorporated by reference in its entirety. Said XML copy, created on Aug. 11, 2023, is named 40848_0077USC1_SL.xml and is 12,672 bytes in size.

### FIELD OF THE INVENTION

The present invention relates to the prevention and/or treatment of allergy and allergic conditions. More specifically, the invention relates to the administration of interleukin-4 receptor (IL-4R) antagonists to prevent or treat allergy in a patient in need thereof.

### BACKGROUND

Allergies and allergic diseases are serious medical conditions with consequences ranging from non-life threatening responses that resolve over time to life threatening effects such as anaphylaxis. Allergic reactions can result from contact or exposure to a variety of products such as certain food items, insect venom, plant-derived material (e.g., pollen), chemicals, drugs/medications, and animal dander. The pathophysiology of allergy is influenced by a complex interplay between Immunoglobulin E (IgE)-mediated sensitization, the immune system, and environmental factors. Current treatment options for allergies include avoidance, pharmacological symptom treatment and prophylaxis using allergen-specific immunotherapies (SIT). Unfortunately, these current treatment strategies are often inadequate, costly, impractical or involve significant risk. For example, avoidance of allergen is not always possible and can negatively impact on patient and caregiver quality of life. Immunotherapeutic approaches, on the other hand, involve deliberate administration of allergen to susceptible individuals and are therefore inherently risky with the potential for unwanted severe allergic reactions or anaphylaxis. Accordingly, an unmet need exists in the art for novel therapeutic approaches that prevent or treat allergies or allergic responses and reduce the risk of developing an allergic response.

### BRIEF SUMMARY OF THE INVENTION

According to certain aspects of the present invention, methods are provided for preventing or treating allergy in a subject. Also included are methods of reducing the susceptibility to an allergic reaction or decreasing allergen sensitization in a subject. In certain embodiments, the invention provides for methods to reduce serum allergen-specific IgE levels in a subject. The methods of the present invention comprise administering to a subject in need thereof a pharmaceutical composition comprising a therapeutically effective amount of an interleukin-4 receptor (IL-4R) antagonist. In certain embodiments, the pharmaceutical composition is administered subcutaneously at a dose of 75-600 mg.

In certain embodiments, the present invention provides methods to prevent or treat allergy, wherein preventing or treating allergy comprises reducing the level of allergen-specific IgE. In certain embodiments, the subject in need thereof exhibits at least a 10%, at least 20%, at least 30%, at least 40%, or at least a 50% decrease in allergen-specific IgE upon administration of the IL-4R antagonist. In certain embodiments, the allergic reaction or susceptibility of a subject to an allergic reaction is triggered by allergen sensitization. In certain embodiments, the present invention provides methods to reduce or abrogate allergen sensitization.

In certain embodiments, the subject is sensitized to an allergen derived from one or more of the following sources including, but not limited to, Alder Grey, Alternaria Tenuis, Bermuda Grass, Silver Birch, Cat Dander, Cladosporium, Cockroach (German), Dermatophagoides farinae (mite), D. pteronyssinus, Dog Dander, Elm, Johnson Grass, White Oak, Ragweed Short, Mugwort Sage, Timothy (Phleum), White Ash, Candida albicans, Malasezzia furfur, Pityrosporum orbiculare, mold, Staphylococcal enterotoxin A, or Staphylococcal enterotoxin B. In certain embodiments, the subject is sensitized to an allergen derived from a food item selected from the group consisting of dairy, fish, shellfish, peanuts, tree nuts, fruit (e.g., melons), egg, wheat, and soy.

According to certain embodiments, the present invention provides methods for treating or preventing allergy or for reducing susceptibility to an allergic reaction in a subject, wherein the methods comprise sequentially administering to the subject about 50 mg to about 600 mg of an IL-4R antagonist as an initial dose followed by one or more secondary doses. In certain embodiments, the initial dose and the one or more secondary doses each comprise about 75 mg to about 300 mg of the IL-4R antagonist. In certain embodiments, the IL-4R antagonist is administered at an initial dose of 400 mg or 600 mg followed by one or more secondary doses wherein each secondary dose comprises 200 mg or 300 mg. According to this aspect of the invention, the pharmaceutical composition may be administered to the subject at a dosing frequency of, e.g., once a week, once in 2 weeks, once in 3 weeks or once in 4 weeks. In one embodiment, the IL-4R antagonist is administered at an initial dose of 400 mg followed by one or more secondary doses wherein each secondary dose comprises 200 mg and is administered weekly.

In certain embodiments, the invention provides methods to treat or prevent allergy, or to reduce susceptibility to an allergic reaction in a subject wherein the subject has a disease or disorder selected from the group consisting of atopic dermatitis, asthma, allergic rhinitis, eosinophilic esophagitis and food allergy. In one embodiment, the subject has moderate-to-severe atopic dermatitis.

Exemplary IL-4R antagonists that can be used in the context of the methods of the present invention include, e.g., small molecule chemical inhibitors of IL-4R or its ligands (IL-4 and/or IL-13), or biological agents that target IL-4R or its ligands. According to certain embodiments, the IL-4R antagonist is an antigen-binding protein (e.g., antibody or antigen-binding fragment thereof) that binds the IL-4Ra chain and blocks signaling by IL-4, IL-13, or both IL-4 and IL-13. In one embodiment, the antibody or antigen-binding fragment thereof that specifically binds IL-4R comprises complementarity determining regions (CDRs) in a heavy chain variable region (HCVR)/light chain variable region (LCVR) sequence pair of SEQ ID NOs: 1/2. In certain embodiments, the antibody or antigen-binding fragment thereof comprises a heavy chain CDR (HCDR1) having amino acid sequence of SEQ ID NO: 3, a HCDR2 having amino acid sequence of SEQ ID NO: 4, a HCDR3 having amino acid sequence of SEQ ID NO: 5, a light chain CDR (LCDR1) having amino acid sequence of SEQ ID NO: 6, a LCDR2 having amino acid sequence LGS, and a LCDR3 having amino acid sequence of SEQ ID NO: 8. One such type of antigen-binding protein that can be used in the context of the methods of the present invention is an anti-IL-4Ra antibody such as dupilumab.

In some embodiments, the pharmaceutical composition is administered subcutaneously or intravenously to the subject.

In certain embodiments, the pharmaceutical composition is administered to the patient before, after or concurrent with a second therapeutic agent. In some embodiments, the second therapeutic agent is selected from the group consisting of another IL-4R inhibitor, an IgE inhibitor, a corticosteroid (e.g., topical corticosteroid or a systemic corticosteroid), a non-steroidal anti-inflammatory drug (NSAID), an anti-histamine, systemic immunotherapy, and IFNγ.

In certain embodiments, the present invention provides use of an IL-4R antagonist of the invention in the manufacture of a medicament to treat or reduce or prevent allergy or allergen sensitization in a patient.

Other embodiments of the present invention will become apparent from a review of the ensuing detailed description.

### BRIEF DESCRIPTION OF THE FIGURES

FIG. 1 shows least squares (LS) mean percent change in Eczema Area Severity Index (EASI) scores from baseline to week 16 in the study described in Example 1. *P <0.0001 vs placebo; qw, once weekly, SE, standard error.

FIG. 2 shows LS mean percent change in peak weekly pruritus numerical rating scale (NRS) scores from baseline to week 16 in the study described in Example 1. *P<0.01 vs placebo; LS, least squares.

FIG. 3 shows serum thymus and activation-regulated chemokine (TARC) levels from baseline to week 16 in the study described in Example 2. **P<0.001 vs placebo; DPL, dupilumab; PBO, placebo; qw, once weekly; SE, standard error.

FIG. 4 shows serum pulmonary and activation-regulated chemokine (PARC) levels from baseline to week 16 in the study described in Example 2. ***P<0.0001 vs placebo; DPL, dupilumab; PBO, placebo; qw, once weekly; SE, standard error.

FIG. 5 shows serum periostin levels from baseline to week 16 in the study described in Example 2. *P<0.01 vs placebo; DPL, dupilumab; PBO, placebo; qw, once weekly; SE, standard error.

FIG. 6 shows serum total IgE levels from baseline to week 16 in the study described in Example 2. ***P<0.0001 vs placebo; DPL, dupilumab; PBO, placebo; qw, once weekly; SE, standard error.

FIG. 7 shows that dupilumab suppresses IgEs specific for a wide variety of allergens in serum after 16-week treatment as described in Example 2.

FIG. 8 shows suppression of IgEs specific for a wide variety of allergens in serum after 16-week treatment of placebo, dupilumab 100 mg every 4 weeks (q4w) or dupilumab 300 mg q4w, as described in Example 2. *P<0.05, **P<0.01, § P<0.001, ¶P<0.0001 vs placebo. D. farinae, Dermatophagoides farinae; D. pteronyssinus, Dermatophagoides pteronyssinus; M. furfur, Malasezzia furfur; P. orbiculare; Pityrosporum orbiculare; S. enterotoxin A/B, Staphylococcal enterotoxin A/B. Interquartile range: Q1, lowest quartile; Q3, upper quartile.

FIG. 9 shows suppression of IgEs specific for a wide variety of allergens in serum after 16-week treatment of dupilumab 200 mg every 2 weeks (q2w), 300 mg q2w, or 300 mg weekly (qw) as described in Example 2. *P<0.05, **P<0.01, § P<0.001, ¶P<0.0001 vs placebo. D. farinae, Dermatophagoides farinae; D. pteronyssinus, Dermatophagoides pteronyssinus; M. furfur, Malasezzia furfur; P. orbiculare; Pityrosporum orbiculare; S. enterotoxin A/B, Staphylococcal enterotoxin A/B. Interquartile range: Q1, lowest quartile; Q3, upper quartile.

FIG. 10 shows Staphylococcus aureus abundance in lesional skin of patients with atopic dermatitis in the study in Example 3.

FIG. 11 shows S. aureus abundance in non-lesional skin of patients with atopic dermatitis in the study in Example 3.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

According to certain aspects, the present invention includes methods for preventing or treating allergy in a subject, wherein the methods comprise administering a therapeutically effective amount of an IL-4R antagonist to the subject in need thereof. As used herein, the terms “treat”, “treating”, or the like, mean to alleviate allergic symptoms, eliminate the causation of allergic symptoms either on a temporary or permanent basis, or to prevent or slow the appearance of allergic symptoms in a subject. As used herein, the terms “prevent”, “preventing”, or the like, refer to preventing development of allergy, an allergic reaction or an allergic condition. The term, as used herein, also includes reducing or abrogating allergen sensitization to prevent an allergic reaction. In some embodiments, the term refers to decreasing the level of serum allergen-specific IgE by at least 10%, at least 20%, at least 30%, at least 40%, or at least 50%, as compared to baseline, upon administration of an IL-4R antagonist as provided by the methods of the present invention.

The present invention includes methods which comprise administering to a subject in need thereof a therapeutic composition comprising an IL-4R antagonist. As used herein, the expression “a subject in need thereof” means a human or non-human animal that exhibits one or more symptoms or indicia of allergy or atopy, and/or who has been diagnosed with allergy to an allergen. In certain embodiments, the term “subject in need thereof” includes subjects that are at an increased risk for developing an allergy or an allergic response to an allergen. In certain embodiments, the term includes subjects that show allergen sensitization to one or more allergens. In certain embodiments, the methods of the present invention may be used to treat subjects that show elevated levels of one or more serum biomarkers including, but not limited to, total IgE, allergen-specific IgE, thymus and activation-regulated chemokine (TARC), pulmonary and activation-regulated chemokine (PARC), lactate dehydrogenase (LDH), and periostin. For example, the methods of the present invention comprise administering an IL-4R antagonist to patients with elevated levels of allergen-specific IgE. The terms “subject” and “patient” have been used interchangeably herein.

As used herein, the terms “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production and/or increased allergen-specific immunoglobulin production.

The term “allergen,” as used herein, includes any substance, chemical, particle or composition which is capable of stimulating an allergic response in a susceptible individual. Allergens may be contained within or derived from a food item such as, e.g., dairy products (e.g., cow's milk), egg, celery, sesame, wheat, soy, fish, shellfish, sugars (e.g., sugars present on meat such as alpha-galactose), peanuts, other legumes (e.g., beans, peas, soybeans, etc.), and tree nuts. Alternatively, an allergen may be contained within or derived from a non-food item such as, e.g., dust (e.g., containing dust mite), pollen, insect venom (e.g., venom of bees, wasps, mosquitos, fire ants, etc.), mold, animal fur, animal dander, wool, latex, metals (e.g., nickel), household cleaners, detergents, medication, cosmetics (e.g., perfumes, etc.), drugs (e.g., penicillin, sulfonamides, salicylate, etc.), therapeutic monoclonal antibodies (e.g., cetuximab), ragweed, grass and birch. Exemplary pollen allergens include, e.g., tree pollens such as birch pollen, cedar pollen, oak pollen, alder pollen, hornbeam pollen, aesculus pollen, willow pollen, poplar pollen, plantanus pollen, tilia pollen, olea pollen, Ashe juniper pollen, and Alstonia scholaris pollen. Other examples of allergens can be found elsewhere herein. The terms “allergen” and “antigen” are used interchangeably through the disclosure.

According to certain aspects, the present invention provides methods to reduce susceptibility to an allergic reaction in a subject, the methods comprising administering a therapeutically effective amount of an IL-4R antagonist to the subject in need thereof. In certain embodiments, the term “subject in need thereof” includes a subject that is susceptible to an allergic reaction or is at an increased risk for developing an allergic reaction to an allergen. In certain embodiments, a subject may be at an increased risk of developing an allergy or an allergic response to an allergen due to sensitization to said allergen. For example, the term includes subjects that show increased levels of serum IgE specific to one or more allergens (“allergen sensitization”). In the context of the present invention, the term “subject in need thereof”, also includes subjects that have a disease or disorder selected from the group consisting of atopic dermatitis, asthma, allergic rhinitis, eosinophilic esophagitis and food allergy. The term “subject” also includes subjects with elevated levels of serum total and allergen-specific IgE, or serum chemokines (e.g., CCL17 or CCL27) that may have an increased risk of developing an allergic response. The present invention provides methods to decrease the risk of developing allergy or allergic response in susceptible subjects.

According to certain aspects, the present invention provides methods of reducing levels of serum allergen-specific IgE in a subject, the methods comprising administering a therapeutically effective amount of an IL-4R antagonist. In certain embodiments, the serum allergen-specific IgE levels are reduced by at least 10%, 20%, 30%, 40% or 50% as compared to the baseline following administration of the IL-4R antagonist.

Methods for detecting and/or quantifying a serum biomarker such as allergen-specific IgE or total IgE are known in the art; kits for measuring such a biomarker are available from various commercial sources; and various commercial diagnostic laboratories offer services which provide measurement of such biomarkers as well.

For example, Phadiatop™ is a commercially available variant of serum specific or antigen-specific IgE assay test that was introduced for the screening of allergic sensitization (Merrett et al 1987, Allergy 17: 409-416). The test provides for simultaneous testing for serum specific IgE to a mixture of relevant allergens causing common inhalant allergies. The test gives a qualitative result, either positive or negative depending upon a fluorescence response obtained. When a patient sample gives a fluorescence response higher than or equal to the reference, a positive test result is indicated. A patient sample with a lower fluorescence response indicates a negative test result. The present invention includes methods comprising selecting a subject who exhibits a positive test result and administering to the subject a therapeutically effective amount of an IL-4R antagonist.

The present invention also includes methods for determining whether a subject is a suitable subject for whom administration of a pharmaceutical composition comprising an IL-4R antagonist would be beneficial. For example, if an individual, prior to receiving a pharmaceutical composition comprising an IL-4R antagonist, exhibits a level of a serum biomarker (e.g., allergen-specific IgE) which signifies allergen sensitization, the individual is therefore identified as a suitable patient for whom administration of a pharmaceutical composition of the invention (a composition comprising an anti-IL-4R antibody) would be beneficial.

According to certain aspects of the invention, methods for preventing or treating allergy are provided which comprise: (a) selecting a subject who exhibits a level of IgE specific to at least one allergen prior to or at the time of treatment which signifies allergic sensitization; and (b) administering to the subject a pharmaceutical composition comprising a therapeutically effective amount of an IL-4R antagonist. In certain embodiments, the patient is selected by determining if the level of allergen-specific IgE is elevated. The level of allergen-specific IgE is determined or quantified by acquiring a sample from the patient for a biomarker assay known in the art. In certain other embodiments, a patient is selected by acquiring information relating to an elevated level of allergen-specific IgE from the patient. In certain embodiments of this aspect of the invention, the subject is selected on the basis of an elevated level of IgE or TARC or periostin.

As will be appreciated by a person of ordinary skill in the art, an increase or decrease in a serum biomarker can be determined by comparing (i) the level of the biomarker measured in a subject at a defined time point after administration of the IL-4R antagonist to (ii) the level of the biomarker measured in the patient prior to the administration of the IL-4R antagonist (i.e., the “baseline measurement”). The defined time point at which the biomarker is measured can be, e.g., at about 4 hours, 8 hours, 12 hours, 1 day, 2 days, 3 days, 4 days, 5 days, 6 days, 7 days, 8 days, 9 days, 10 days, 15 days, 20 days, 35 days, 40 days, 50 days, 55 days, 60 days, 65 days, 70 days, 75 days, 80 days, 85 days, 100 days, 150 days, or more after administration of the IL-4R antagonist.

According to certain particular embodiments of the present invention, a subject may exhibit a decrease in the level of serum IgE specific to one or more allergens following administration of a pharmaceutical composition comprising an IL-4R antagonist (e.g., an anti-IL-4R antibody). For example, at about day 8, day 15, day 22, day 25, day 29, day 36, day 43, day 50, day 57, day 64, day 71, day 85, or day 112, following administration of one or more doses of a pharmaceutical composition comprising about 75, 150, 200 or 300 mg of an anti-hlL-4R antibody (e.g., dupilumab), the subject, according to the present invention, may exhibit a decrease in allergen-specific IgE of about 1%, 2%, 5%, 10%, 15%, 20%, 25%, 30%, 35%, 40%, 45%, 50%, 55%, 60%, 65%, 70%, 75%, 80%, 85%, 90% or more from baseline (wherein “baseline” is defined as the level of allergen-specific IgE in the subject just prior to the first administration).

### Interleukin-4 Receptor Antagonists

The methods of the present invention comprise administering to a subject in need thereof a therapeutic composition comprising an interleukin-4 receptor (IL-4R) antagonist. As used herein, an “IL-4R antagonist” (also referred to herein as an “IL-4R inhibitor,” an “IL-4Rα antagonist,” an “IL-4R blocker,” an “IL-4Rα blocker,” etc.) is any agent which binds to or interacts with IL-4Rα or an IL-4R ligand, and inhibits or attenuates the normal biological signaling function a type 1 and/or a type 2 IL-4 receptor. Human IL-4Rα has the amino acid sequence of SEQ ID NO: 11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present invention may function by blocking IL-4-mediated signaling, IL-13-mediated signaling, or both IL-4- and IL-13-mediated signaling. The IL-4R antagonists of the present invention may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

According to certain exemplary embodiments of the present invention, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized.

The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (iX) V_{L}-C_{H}2; (X) V_{L}-C_{H}3; (Xi) V_{L}-C_{H}1-C_{H}2; (Xii) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present invention using routine techniques available in the art. For example, the present invention includes methods comprising the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind IL-4Rα. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4Rα, as used in the context of the present invention, includes antibodies that bind IL-4Rα or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay. An isolated antibody that specifically binds human IL-4Rα may, however, have cross-reactivity to other antigens, such as IL-4Rα molecules from other (non-human) species.

According to certain exemplary embodiments of the present invention, the IL-4R antagonist is an anti-IL-4Rα antibody, or antigen-binding fragment thereof comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693. In certain exemplary embodiments, the anti-IL-4Rα antibody or antigen-binding fragment thereof that can be used in the context of the methods of the present invention comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-IL-4Rα antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence LGS; and the LCDR3 comprises the amino acid sequence of SEQ ID NO: 8. In some embodiments, the anti-IL-4R antibody or antigen-binding fragment thereof comprises an HCVR comprising SEQ ID NO: 1 and an LCVR comprising SEQ ID NO: 2. According to certain exemplary embodiments, the methods of the present invention comprise the use of the anti-IL-4R antibody comprising HCDR1-HCDR2-HCDR3-LCDR1-LCDR2-LCDR3 amino acid sequences (of) SEQ ID NO: 3-SEQ ID NO: 4-SEQ ID NO: 5-SEQ ID NO: 6-LGS-SEQ ID NO: 8 (referred to and known in the art as “dupilumab”), or a bioequivalent thereof. In certain embodiments, the methods of the present invention comprise the use of an anti-IL-4R antibody, wherein the antibody comprises a heavy chain comprising the amino acid sequence of SEQ ID NO: 9. In some embodiments, the anti-IL-4R antibody comprises a light chain comprising the amino acid sequence of SEQ ID NO: 10. An exemplary antibody comprising a heavy chain comprising the amino acid sequence of SEQ ID NO: 9 and a light chain comprising the amino acid sequence of SEQ ID NO: 10 is the fully human anti-IL-4R antibody known as dupilumab. According to certain exemplary embodiments, the methods of the present invention comprise the use of dupilumab, or a bioequivalent thereof. The term “bioequivalent”, as used herein, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In the context of the invention, the term refers to antigen-binding proteins that bind to IL-4R which do not have clinically meaningful differences with dupilumab in their safety, purity and/or potency.

Other anti-IL-4Rα antibodies that can be used in the context of the methods of the present invention include, e.g., the antibody referred to and known in the art as AMG317 (Corren et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, or U.S. Pat. No. 8,877,189.

The anti-IL-4Rα antibodies used in the context of the methods of the present invention may have pH-dependent binding characteristics. For example, an anti-IL-4Rα antibody for use in the methods of the present invention may exhibit reduced binding to IL-4Rα at acidic pH as compared to neutral pH. Alternatively, an anti-IL-4Rα antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Pharmaceutical Compositions

The present invention includes methods which comprise administering an IL-4R antagonist to a patient, wherein the IL-4R antagonist (e.g., an anti-IL-4R antibody) is contained within a pharmaceutical composition. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient according to the methods of the present invention may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351). Specific exemplary doses of anti-IL4R antibodies, and administration regimens involving the same, that can be used in the context of the present invention are disclosed elsewhere herein.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present invention are disclosed, e.g., in U.S. Pat. No. 8,945,559.

### Dosage

The amount of IL-4R antagonist (e.g., anti-IL-4R antibody) administered to a subject according to the methods of the present invention is, generally, a therapeutically effective amount. As used herein, the phrase “therapeutically effective amount” means an amount of IL-4R antagonist that results in one or more of: (a) prevention of allergy; (b) treatment of or reduction in the severity of an allergic reaction; (c) reduction in the level of serum allergen-specific IgE; (d) reduction of allergen sensitization; and/or (e) reduction in susceptibility to an allergic reaction.

In the case of an anti-IL-4R antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In certain embodiments, 75 mg, 100 mg, 150 mg, 200 mg, or 300 mg of an anti-IL-4R antibody is administered to a subject.

The amount of IL-4R antagonist contained within the individual doses may be expressed in terms of milligrams of antibody per kilogram of patient body weight (i.e., mg/kg). For example, the IL-4R antagonist may be administered to a patient at a dose of about 0.0001 to about 10 mg/kg of patient body weight.

### Combination Therapies

The methods of the present invention, according to certain embodiments, comprise administering to the subject one or more additional therapeutic agents in combination with the IL-4R antagonist. As used herein, the expression “in combination with” means that the additional therapeutic agents are administered before, after, or concurrent with the pharmaceutical composition comprising the IL-4R antagonist. The term “in combination with” also includes sequential or concomitant administration of IL-4R antagonist and a second therapeutic agent.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

The additional therapeutic agent may be, e.g., another IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), an IL-13 antagonist, a tumor necrosis factor (TNF) antagonist, an IL-8 antagonist, an IL-9 antagonist, an IL-17 antagonist, an IL-5 antagonist, an IgE antagonist, a CD48 antagonist, an IL-31 antagonist (including, e.g., as set forth in U.S. Pat. No. 7,531,637), a thymic stromal lymphopoietin (TSLP) antagonist (including, e.g., as set forth in US 2011/027468), interferon-gamma (IFNγ) antibiotics, topical corticosteroids, tacrolimus, pimecrolimus, cyclosporine, azathioprine, methotrexate, cromolyn sodium, proteinase inhibitors, systemic corticosteroids, systemic immunotherapy, anti-histamines, or combinations thereof. In certain embodiments, the pharmaceutical composition comprising an anti-IL4R antagonist is administered to a subject in conjunction with a non-pharmaceutical therapy such as ultraviolet (UV) light therapy.

### Administration Regimens

The present invention includes methods comprising administering to a subject a pharmaceutical composition comprising an IL-4R antagonist at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments involving the administration of a pharmaceutical composition comprising an anti-IL-4R antibody, once a week dosing at an amount of about 75 mg, 150 mg, 200 mg, or 300 mg, can be employed.

According to certain embodiments of the present invention, multiple doses of an IL-4R antagonist may be administered to a subject over a defined time course. The methods according to this aspect of the invention comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks or months). The present invention includes methods which comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an IL-4R antagonist may be administered to a patient with AD at a loading dose of about 400 mg or about 600 mg followed by one or more maintenance doses of about 75 mg to about 300 mg. In one embodiment, the initial dose and the one or more secondary doses each include 50 mg to 600 mg of the IL-4R antagonist, e.g., 100 mg to 400 mg of the IL-4R antagonist, e.g., 100 mg, 150 mg, 200 mg, 250 mg, 300 mg, 400 mg or 500 mg of the IL-4R antagonist. In some embodiments, the initial dose and the one or more secondary doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist.

In one exemplary embodiment of the present invention, each secondary and/or tertiary dose is administered 1 to 14 (e.g., 1, 1 1/2, 2, 2 1/2, 3, 3 1/2, 4, 4 1/2, 5, 5 1/2, 6, 6 1/2, 7, 7 1/2, 8, 8 1/2, 9, 9 1/2, 10, 10 1/2, 11, 11 1/2, 12, 12 1/2, 13, 13 1/2, 14, 14 1/2, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods according to this aspect of the invention may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial Investigating Efficacy of Dupilumab in Adult Patients with Moderate-to-Severe AD

### Study Design and Objectives

This was a 32-week randomized, double-blind, placebo-controlled, parallel group study to assess the safety, efficacy, biomarker profile, functional concentrations and immunogenicity of dupilumab administered weekly for 16 consecutive weeks to adult patients with moderate-to-severe AD. Dupilumab is a fully human anti-IL-4R antibody comprising a heavy chain comprising the amino acid sequence of SEQ ID NO: 9 and a light chain comprising the amino acid sequence of SEQ ID NO: 10; an HCVR/LCVR amino acid sequence pair comprising SEQ ID NOs: 1/2; and heavy and light chain CDR sequences comprising the amino acid sequences (of) SEQ ID NO: 3, SEQ ID NO: 4, SEQ ID NO: 5, SEQ ID NO: 6, LGS, AND SEQ ID NO: 8.

Eligible patients were randomized in a 1:1 ratio to receive subcutaneous (SC) dupilumab or SC placebo. Randomization was stratified by disease severity (moderate vs. severe AD). After providing informed consent, patients were assessed for study eligibility at the screening visit. Patients who met eligibility criteria underwent day 1/baseline assessments, randomization and received a loading dose (400 mg SC of study drug) with subsequent weekly injections of study drug (200 mg SC) from week 1 through week 15. During this time, patients underwent weekly assessments, most through clinical visits, but some through telephone contact. Depending upon the patient's preference and capabilities, patients and/or caregivers were trained at the study site on injecting the study drug at the first 5 treatment visits (visits 2, 3, 4, 5 and 6) and subsequently administered the study drug outside the clinic at visits 7, 9, 11, 13 and 15, which required only a telephone contact. Safety, laboratory and clinical effect assessments were performed at specific clinic visits. The end of treatment period visit occurred at week 16 after the last dose of study drug, when the primary endpoint was assessed. Follow-up visits occurred every 2 weeks from week 18 through week 32. The end of study visit occurred at week 32.

Rescue treatment for AD (medication and/or phototherapy) was provided to the patients, if necessary. Patients who needed rescue treatment were discontinued from the study treatment, but continued to follow the schedule of study assessments. Efficacy measurements (e.g., IGA, EASI, etc.) were obtained before any rescue treatment was administered.

Samples for clinical chemistry and hematology, drug concentration and anti-drug antibodies were collected at various time-points during the study. In addition, 1 sample for DNA analysis and multiple samples for RNA analysis were collected.

Treatment assignment was allocated randomly, to avoid predisposition in assigning patients to a particular treatment group and to minimize systematic differences between treatment groups with respect to baseline variables that could affect the outcome. The double-blind design was intended to minimize any potential bias in clinical assessments and patient reported outcomes resulting from investigator's or patient's knowledge of treatment allocation. The placebo arm provided a reliable reference of any apparent effects of the study treatment. Patients assigned to the dupilumab arm received 200 mg once weekly (qw) after a 400 mg loading dose on day 1. Study treatment was administered for 16 weeks so as to stabilize systemic concentrations of functional dupilumab. After the treatment, all patients were followed for 16 weeks (i.e., approximately 5 half-lives) to ensure that dupilumab clearance was virtually complete (plasma concentrations below the lower limit of quantification) before the end of study visit.

The primary objective of the study was to assess the efficacy of dupilumab, compared to placebo, in adult patients with moderate-to-severe AD.

The secondary objectives of the study were: (1) to assess the safety of dupilumab, compared to placebo; (2) to assess the concentration of dupilumab; and (3) to assess the potential anti-drug antibody response to dupilumab, compared to placebo, in adult patients with moderate-to-severe AD.

The exploratory objectives were: (1) to assess the effect of dupilumab on epidermal hyperplasia; (2) to assess the pharmacodynamic (PD) effect of dupilumab on biomarkers over time; and (3) to assess the predictive value of thymus and activation-regulated cytokine (TARC) on the EASI response.

The target population included adults with moderate-to-severe AD which was not adequately controlled with topical medications or where the topical treatment was inadvisable (due to, e.g., side effects or safety risks). Eligible patients were randomized in a 1:1 ratio to receive subcutaneous (SC) dupilumab or SC placebo. Randomization was stratified by disease severity (moderate vs. severe AD). Patients received a loading dose (400 mg SC) of study drug on day 1, followed by weekly injections of study drug (200 mg SC) from week 1 through week 15. Patients were required to apply a topical emollient twice daily from day −7 through day 8. Safety, laboratory and clinical effect assessments were performed at specified clinic visits. Samples for clinical chemistry and hematology, drug concentration and anti-drug antibodies were collected at various time-points throughout the study. In addition, 1 sample for DNA analysis and multiple samples for RNA analysis were collected. The end of treatment period visit occurred at week 16, 1 week after the last dose of study drug, when the primary endpoint was assessed. Follow-up visits occurred every 2 weeks from week 18 through week 32. The end of study visit occurred at week 32.

The primary endpoint in the study was the percent change in EASI score from baseline to week 16. The secondary endpoints included: (1) proportion of patients achieving IGA 0 (clear) or 1 (almost clear) at week 16; (2) proportion of patients achieving IGA score reduction of >2 at week 16; (3) absolute and percent change from baseline in pruritus scores (NRS and 4-point categorical scale); (4) absolute change in EASI scores from baseline to week 16; (5) absolute and percent change in SCORAD scores from baseline to week 16; (6) proportion of patients achieving EASI-50, EASI-75 and EASI-90 (50, 75 and 90% reduction from baseline in EASI score) at week 16; (7) proportion of patients achieving SCORAD-50, SCORAD-75 and SCORAD-90 (50, 75 and 90% reduction from baseline in SCORAD score) at week 16; (8) absolute and percent change from baseline in POEM scores; (9) changes from baseline in GISS components (erythema, infiltration/population, excoriations, and lichenification); (10) changes from baseline in GISS cumulative score; (11) incidence of TEAEs from baseline through week 32; and (12) dupilumab serum concentrations overtime from baseline through week 32.

The exploratory endpoints include: (1) the proportion of patients with a histological response consisting of significantly decreased epidermal hyperplasia in lesional skin and defined as >40% reduction from baseline in epidermal thickness and/or reversal of K16 expression by immunohistochemistry; (2) change in TARC from baseline through week 16; (3) change in IgE from baseline through week 16; (4) change in allergen-specific IgE through week 16; and (5) correlation of baseline TARC and IgE on EASI response.

### Patient Selection

The target population included adults with moderate-to-severe AD which was not adequately controlled with topical medications or for whom topical treatment is otherwise inadvisable (due to e.g., side effects or safety risks).

Inclusion Criteria: A patient had to meet the following criteria to be eligible for inclusion in the study: (1) male or female, 18 years or older; (2) chronic AD, that had been present for at least 3 years before the screening visit; (3) EASI score ≥12 at the screening visit and ≥16 at the baseline visit; (4) IGA score 3 (on the 0-4 IGA scale) at the screening and baseline visits; (5) ≥10% BSA of AD involvement at the screening and baseline visits; (6) patients with documented recent history (within 6 months before the screening visit) of inadequate response to outpatient treatment with topical medications or for whom topical treatments were otherwise inadvisable (e.g., because of important side effects or safety risks) [For the purpose of this protocol, inadequate response represented failure to achieve and/or maintain remission or a low disease activity state (e.g., IGA 0=clear to 2=mild) despite treatment with topical corticosteroids of medium to high potency (±topical calcineurin inhibitors as appropriate]. To assess inadequacy of response to intensive treatment, topical treatment was applied daily for at least 28 days or for the maximum duration recommended by the product prescribing information (e.g., 14 days for super-potent topical corticosteroids), whichever was shorter. Following intensive daily treatment, inadequacy of response was determined based on failure to maintain a low disease activity state despite applications of topical medications on a less intensive maintenance schedule (i.e., 2 days per week). Important side effects or safety risks that outweighed the potential treatment benefits (e.g., hypersensitivity reactions, significant skin atrophy, systemic effects, etc., or imminence thereof), as assessed by the investigator or by patient's treating physician. Acceptable documentation included contemporaneous chart notes that recorded prescription of topical corticosteroids and/or topical calcineurin inhibitors, and treatment outcome, investigator documentation based on communication with patient's treating physician, or medical history provided by the patient in the event that other forms of documentation were not available (e.g., patient had not seen a physician for AD in the last 6 months)]; (7) patients must have applied a stable dose of topical emollient (moisturizer) twice daily for at least 7 days before the baseline visit; (8) willing and able to comply with all clinic visits and study-related procedures; (9) able to understand and complete study-related questionnaires; and (10) provide signed informed consent.

Exclusion Criteria: The following were the exclusion criteria for the study: (1) prior participation in a clinical trial with dupilumab; (2) treatment with an investigational drug within 8 weeks or within 5 half-lives (if known), whichever was longer, before the baseline visit; (3) the following treatments within 4 weeks before the baseline visit, or any condition that, in the opinion of the investigator, would require such treatment(s) during the first 4 weeks of study treatment—systemic corticosteroids, immunosuppressive/immunomodulating drugs (e.g., cyclosporine, mycophenolate-mofetil, IFNγ, Janus kinase (JAK) inhibitors, azathioprine or methotrexate), or phototherapy for AD; (4) treatment with topical corticosteroids, tacrolimus and/or pimecrolimus within 1 week before the baseline visit; (5) treatment with biologics as follows: any cell-depleting agents including but not limited to rituximab: within 6 months before the baseline visit, or until lymphocyte and CD 19+ lymphocyte count returned to normal, whichever was longer; infliximab, adalimumab, golimumab, certolizumab pegol, abatacept, etanercept, anakinra: within 16 weeks before the baseline visit for any indication, or within 5 years for dermatological indications, other biologics: within 5 half-lives (if known) or 16 weeks, whichever was longer, before the baseline visit; (6) initiation of treatment of AD with prescription moisturizers or moisturizers containing additives such as ceramide, hyaluronic acid, urea, or filaggrin during the screening period (patients could continue using stable doses of such moisturizers if initiated before the screening visit); (7) regular use (more than 2 visits per week) of a tanning booth/parlor within 4 weeks before the baseline visit; (8) planned or anticipated use of any prohibited medications and procedures during study treatment; (9) treatment with a live (attenuated) vaccine within 12 weeks before the baseline visit; (10) chronic or acute infection requiring treatment with systemic antibiotics, antivirals, antiparasitics, antiprotozoals, or antifungals within 4 weeks before the screening visit, or superficial skin infections within 1 week before the screening visit; (11) known or suspected immunosuppression, including history of invasive opportunistic infections (e.g., tuberculosis, histoplasmosis, listeriosis, coccidioidomycosis, pneumocystosis, aspergillosis) despite infection resolution, or otherwise recurrent infections of abnormal frequency, or prolonged infections suggesting an immune-compromised status; (12) known history of human immunodeficiency virus (HIV) infection or HIV seropositivity at the screening visit; (13) positive or indeterminate hepatitis B surface antigen (HBsAg), hepatitis B core antibody (HBcAb), or hepatitis C antibody at the screening visit; (14) elevated transaminases (ALT and/or AST) more than 3 times the upper limit of normal (>3 xULN) at the screening visit; (15) history of clinical endoparasitosis within 12 months before the baseline visit, other than treated vaginal trichomoniasis; (16) presence of skin comorbidities that could interfere with study assessments; (17) history of malignancy within 5 years before the baseline visit, except completely treated in situ carcinoma of the cervix, and completely treated and resolved non-metastatic squamous or basal cell carcinoma of the skin; (18) history of non-malignant lymphoproliferative disorders; (19) high risk of parasite infection, such as residence within or recent travel (within 12 months before the baseline visit) to areas endemic for endoparasitoses, where the circumstances were consistent with parasite exposure (e.g., extended stay, rural or slum areas, lack of running water, consumption of uncooked, undercooked, or otherwise potentially contaminated food, close contact with carriers and vectors, etc.), unless subsequent medical assessments (e.g., stool exam, blood tests, etc.) had ruled out the possibility of parasite infection/infestation; (20) history of alcohol or drug abuse within 2 years before the screening visit; (21) severe concomitant illness(es) that, would adversely affect the patient's participation in the study. Examples included, but were not limited to patients with short life expectancy, patients with uncontrolled diabetes (HbAlc >9%), patients with cardiovascular conditions (e.g., stage Ill or IV cardiac failure according to the New York Heart Association classification), severe renal conditions (e.g., patients on dialysis) hepato-biliary conditions (e.g., Child-Pugh class B or C), neurological conditions (e.g., demyelinating diseases), active major autoimmune diseases (e.g., lupus, inflammatory bowel disease, rheumatoid arthritis, etc.), other severe endocrinological, gastrointestinal, metabolic, pulmonary, or lymphatic diseases; (22) any other medical or psychological condition including relevant laboratory abnormalities at screening that suggested a new and/or insufficiently understood disease, could present an unreasonable risk to the study patient as a result of his/her participation in this clinical trial, could make patient's participation unreliable, or interfere with study assessments. This included hypersensitivity to local anesthetics, bleeding disorders, treatment with anticoagulants or other conditions that could make the biopsy procedure inadvisable; (23) planned major surgical procedure during the patient's participation in this study; (24) pregnant or breast-feeding women or women planning to become pregnant or breastfeed during the study; and (25) women unwilling to use adequate birth control, if of reproductive potential and sexually active.

### Study Treatments

Patients received a subcutaneous loading dose of 400 mg dupilumab on day 1 followed by 200 mg weekly (qw) from week 1 through week 15. Patients on placebo received a loading dose at day 1 followed by weekly subcutaneous dose of placebo from week 1 to week 15. Patients were required to apply a topical emollient twice daily from day −7 through day 8.

### Procedures and Assessments

The efficacy of dupilumab in this population was assessed by AD disease severity scores, quality of life (QOL) questionnaires, pruritus assessments, and patient-reported outcomes. AD severity scores included AD-associated clinical parameters such as Eczema Area and Severity Index (EASI), Investigator's Global Assessment (IGA), Pruritus Numerical Rating Scale (NRS), Body Surface Area (BSA), 5-D Pruritus, SCORing Atopic Dermatitis (SCORAD), Patient Oriented Eczema Measure (POEM), and Global Individual Sign Score (GISS), which are described in US Patent Application Publication No. US2014/0072583 (incorporated by reference herein in its entirety). Quality of Life (QOL) questionnaires included Patient Global Assessment of Disease Status, Dermatology Life Quality Index (DLQI), POEM, EQ-5D, Itchy QoL, and Hospital Anxiety and Depression Scale (HADS), described in US2014/0072583 (incorporated by reference in its entirety). Skin barrier function tests, photographs of the AD area and skin swab samples for exploratory microbiome analyses were also collected. The safety of dupilumab in this population was assessed by evaluating TEAEs, detailed medical history, thorough physical examination, vital signs, electrocardiogram (ECG), and clinical laboratory testing. Concomitant medications and procedures were collected from time of informed consent to the end of the study. Blinded safety data was reviewed on an ongoing basis. Blood samples were collected for drug concentration and anti-dupilumab antibody levels at pre-determined time points. Research samples and samples for exploratory biomarker analysis were collected. Skin biopsy samples were also collected for exploratory biomarker analysis.

### Statistical Analyses

Primary and secondary continuous variables were analyzed using an analysis of covariance (ANCOVA) model with treatment and randomization strata (moderate vs. severe), and relevant endpoint baseline values as covariates. The efficacy data were set to missing after rescue medication was used or after the patient discontinued from the study. Then all missing values were imputed using the last observation carried forward (LOCF) method. EASI and pruritus NRS were reported as least squares (LS) mean (standard error [SE]) percent changes from baseline to Week 16, derived from the LOCF approach.

### Results

Patient Disposition and Baseline Characteristics: 54 patients were randomized to placebo (n=27) or dupilumab 200 mg qw (n=27). Baseline demographics and clinical characteristics were balanced between the treatment groups (Table 1). More than 75% of patients had used prior medications including antihistamines, topical corticosteroids (of groups I, II and III, by potency), and drugs for obstructive airway diseases. A higher proportion of patients on placebo used corticosteroids than patients on dupilumab.

TABLE 1
Baseline patient demographics and clinical
characteristics by treatment group

| | Placebo | | Dupilumab | |
|---|---|---|---|---|
| | (n = 27) | | 200 mg (n = 27) | |
| Variable | | | | |
| Age, median (IQR), years | 43 | (20, 82) | 35 | (18, 71) |
| Male sex, n (%) | 14 | (51.9) | 16 | (59.3) |
| AD duration, mean ± SD, years | 35.4 ± 16.3 | | 26.3 ± 17.21 | |
| BSA, mean ± SD, % | 54.5 ± 26.91 | | 53.8 ± 29.72 | |
| EASI score (0-72), mean ± SD | 34.2 ± 14.59 | | 33.4 ± 15.41 | |
| Total SCORAD score (0-103), | 65.1 ± 13.36 | | 64.2 ± 17.67 | |
| mean ± SD | | | | |
| Peak Pruritus NRS score (0-10), | 7.4 ± 2.04 | | 7.1 ± 2.42 | |
| mean ± SD | | | | |
| IGA score (0-4), n (%) | | | | |
| Score = 3 (moderate) | 13 | (48.1) | 14 | (51.9) |
| Score = 4 (severe) | 14 | (51.9) | 13 | (48.1) |

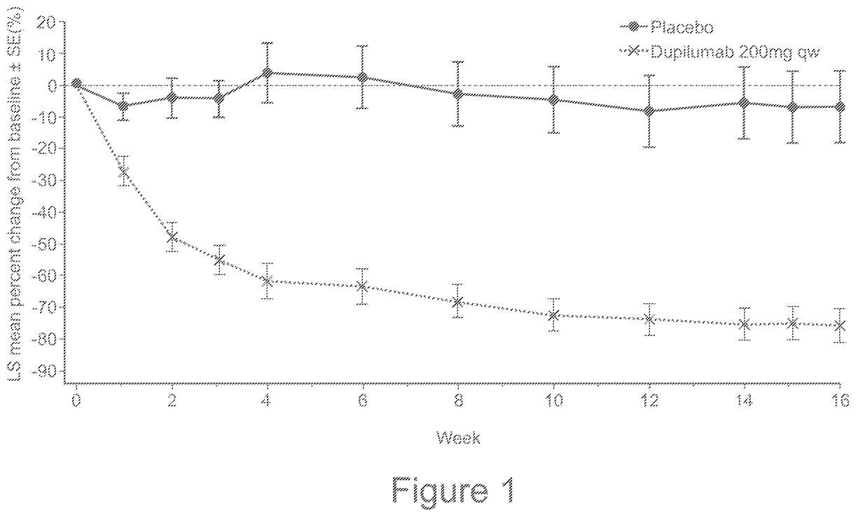

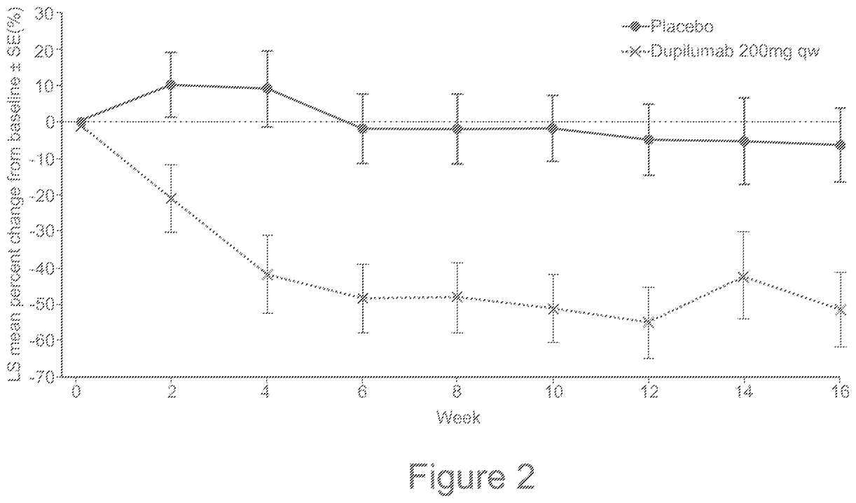

Efficacy: Dupilumab treatment significantly improved (reduced) EASI scores from baseline to Week 16 compared with placebo (SE): −75.2% (8.15) vs −5.8% (8.16); P<0.0001 (FIG. 1). Peak weekly pruritus NRS was also significantly reduced from baseline to Week 16 with dupilumab treatment compared with placebo (SE): −51.5% (10.2) vs −6.3% (10.0); P=0.0027 (FIG. 2). A greater proportion of the dupilumab group patients [14/27 (51.9%)] achieved a reduction in IGA of >2 points by week 16, compared with the placebo group [1/27 (3.7%)]. 37% of dupilumab group patients achieved a score of 0 (clear) or 1 (almost clear) by week, compared with no patient in the placebo group. The proportions of patients who achieved reduction in their EASI score by week 16 were consistently greater in the dupilumab group, than in the placebo group, as evidenced by the proportions of patients with EASI-50 (21/27 [77.8%] dupilumab versus 6/27 [22.2%] placebo, p<0.0001), EASI-75 (18/27 [66.7%] dupilumab versus 4/27 [14.8%] placebo, p=0.0001), and EASI-90 (9/27 [33.3%] dupilumab versus 0/27 [0%] placebo, p=0.0011). The LS mean (±SE) percentage change in SCORAD score from baseline to week 16 was consistent with the change in absolute mean SCORAD (−54.8±5.40% dupilumab versus −8.2±5.41%, p<0.0001). The proportions of patients who achieved a 50% SCORAD reduction (SCORAD-50) by week 16 were observed to be greater in the dupilumab group than in placebo group (15/27[55.6%] dupilumab versus 2/27 [7.4%] placebo, p=0.0002). The dupilumab group showed LS mean (±SE) percentage decrease from baseline in BSA involvement from baseline to week 16 of −69.0±12.61%, whereas the placebo group showed an increase of 13.6±12.61%.

Safety: Dupilumab was safe and well tolerated and had an acceptable safety profile. 23/27 (85.2%) patients in the dupilumab group and 24/27 (88.9%) patients in the placebo group had at least 1 TEAE. Serious TEAEs were reported in 3/27 (11.1%) patients in the placebo group and none were reported in the dupilumab group. Most TEAEs were of mild or moderate severity. Common TEAEs (by Medical Dictionary for Regulatory Activities [MedDRA] Preferred Term) included nasopharyngitis (dupilumab: 3/27 [11.1%] of patients; placebo: 5/27 [18.5%] of patients), upper respiratory tract infection (4/27 [14.8%] and 4/27 [14.8%], respectively), viral upper respiratory tract infection (3/27 [11.1%] and 2/27 [7.4%], respectively) and injection-site reactions (by MedDRA High Level Term; 5/27 [18.5%] and 1/27 [3.7%], respectively).

### Example 2: Biomarker Analyses

### A. Study A

In Study “A”, serum biomarkers were measured in samples from a clinical trial involving subjects with moderate-to-severe atopic dermatitis (AD). Subjects with AD were administered 16 weekly doses of either dupilumab (200 mg) or placebo; patients on dupilumab received a loading dose of 400 mg on day 1. AD-associated serum biomarkers such as thymus and activation-regulated chemokine (TARC), pulmonary and activity-regulated chemokine (PARC), periostin, lactate dehydrogenase (LDH), eosinophil, total IgE, and antigen-specific IgE are described in US Patent Application Publication No. US2014/0072583 (incorporated by reference in its entirety). Serum biomarkers were measured at various time points between screening and Week 32 and included: Serum TARC (Human CCL17/TARC Quantikine ELISA kit; R&D Systems, Minneapolis, MN, USA) and periostin (Human Periostin/OSF-2 DuoSet 15 Plate; R&D Systems). Total IgE and allergen-specific IgEs in serum were measured by ImmunoCap assay (ImmunoCAPR Fluorescent Enzyme Immunoassay; Thermo Scientific, Uppsala, Sweden). Allergen IgE panels were established for common aeroallergens for the region together with S. aureus enterotoxin A and B IgEs. For allergen-specific IgE, the lower limit of quantitation was 0.10 kU/L; a level >0.35 kU/L was considered evidence of allergen sensitization.

Exploratory variables, serum biomarkers TARC, PARC, periostin and total IgE were plotted as mean (SE) percent changes from baseline; antigen-specific IgEs abundance were reported as median (interquartile range [IQR]) percent change from baseline. Data were set to missing after rescue medication. Variables were not adjusted for multiplicity and thus nominal p values are provided.

TABLE 2
Baseline Biomarker Scores

| | | | Dupilumab | |
|---|---|---|---|---|
| | Placebo | | 200 mg | |
| Variable | (n = 27) | | (n = 27) | |
| Serum TARC, mean (SD), pg/mL | 11360 | (24670) | 7722 | (13590) |
| Serum PARC, mean (SD), ng/mL | 206 | (188) | 168 | (127) |
| Serum periostin, mean (SD), ng/mL | 147 | (100) | 154 | (117) |
| Serum total IgE, mean (SD), IU/mL | 5641 | (4706) | 3868 | (4248) |

TARC, thymus and activation-regulated chemokine;
SD, standard deviation;
PARC, pulmonary and activation-regulated chemokine

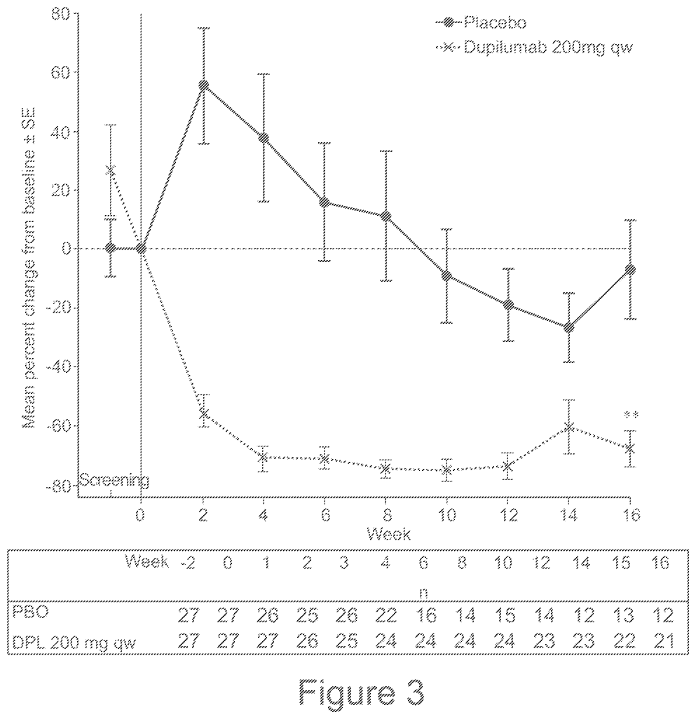

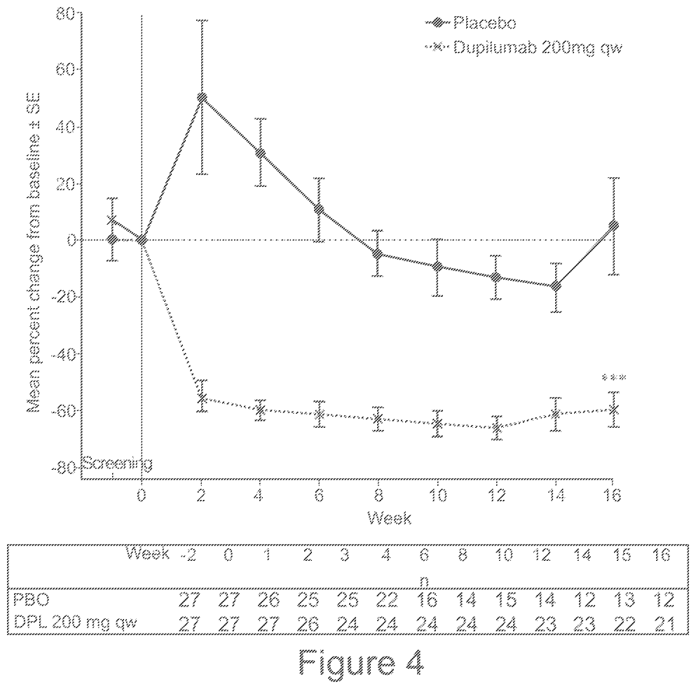

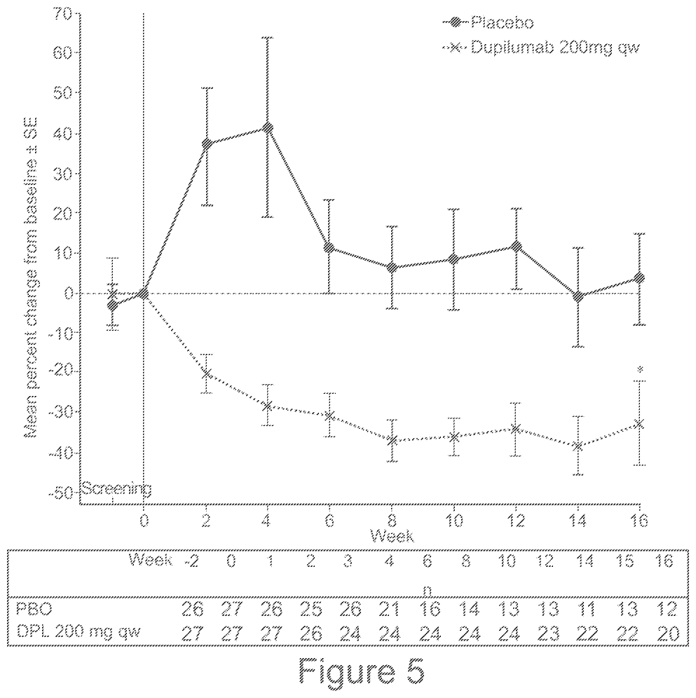

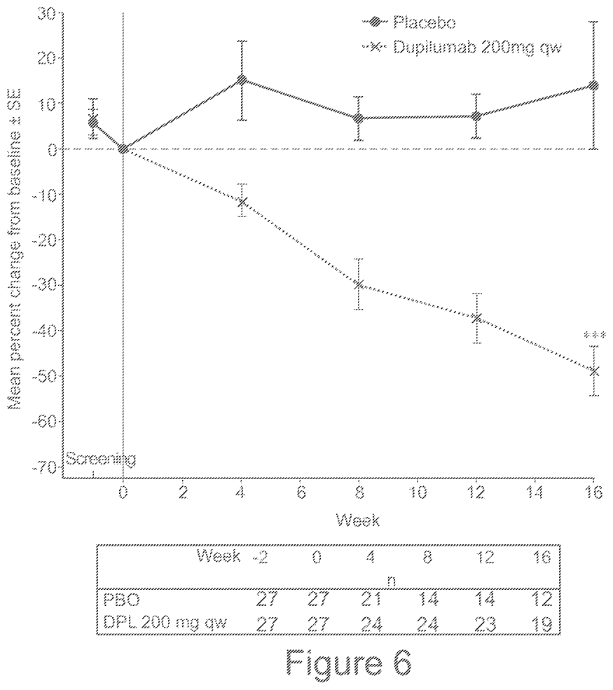

Table 2 shows the baseline biomarker scores in both the treatment groups. Rapid and significant decreases in serum TARC, PARC and periostin (FIGS. 3-5) were observed with dupilumab treatment compared with placebo. Serum total IgE levels steadily declined during the treatment period, demonstrating a significant reduction at Week 16 in the dupilumab group compared with placebo (FIG. 6).

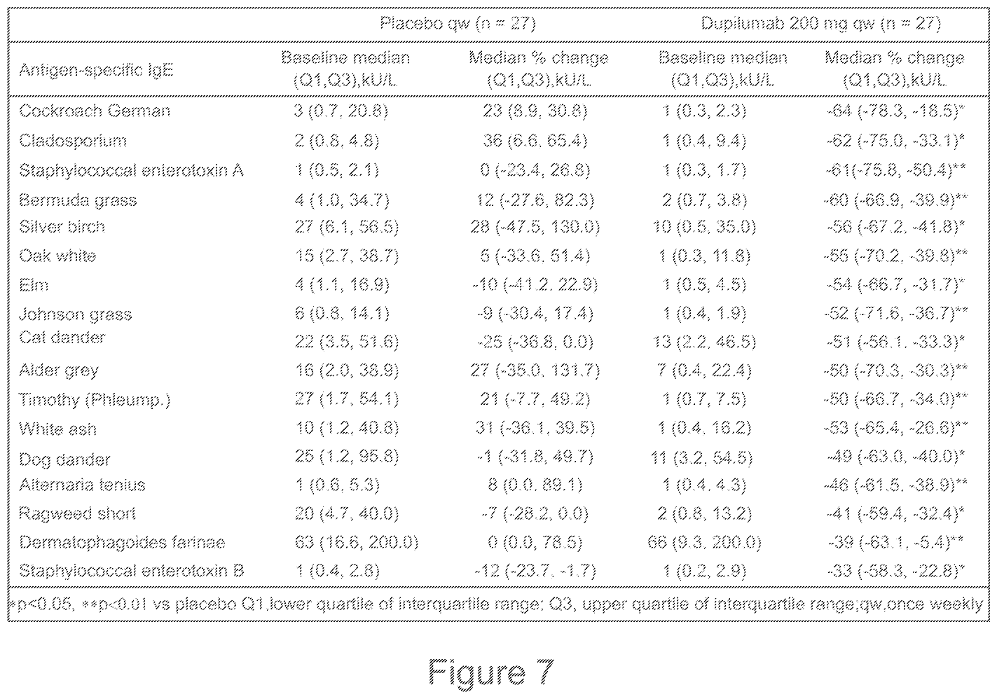

Mean and median decreases from baseline in antigen-specific IgEs, from week 1 through the end of the study (week 32) were noted for IgEs elicited against all allergen panels tested, which comprised Alder Grey, Alternaria Tenuis, Bermuda Grass, Silver Birch, Cat Dander, Cladosporium, Cockroach (German), Dermatophagoides farinae (mite), Dog Dander, Elm, Johnson Grass, White Oak, Ragweed Short, Mugwort Sage, Timothy (Phleum), White Ash, Staphylococcal enterotoxin A, and Staphylococcal enterotoxin B. Dupilumab significantly suppressed a wide range of serum allergen-specific IgEs compared with placebo (FIG. 7; median baseline (IQR) and median percent change (IQR) from baseline to Week 16).

### B. Study B

In “Study B”, serum biomarkers were measured in samples from a clinical trial involving subjects with moderate-to-severe AD. Subjects with AD were randomized in a 1:1:1:1:1:1 ratio to receive 16 weeks of treatment with subcutaneous placebo weekly; or dupilumab 100 mg every 4 weeks (q4w), 300 mg q4w, 200 mg every 2 weeks (q2w), 300 mg q2w, or 300 mg weekly (qw). Patients in the 300 mg dose groups received a loading dose of 600 mg, whilst patients in the 200 and 100 mg dose groups received 400 mg on Day 1. The 16-week treatment period was followed by a 16-week safety follow-up (32-week total study period). AD-associated serum biomarkers such as thymus and activation-regulated chemokine (TARC), pulmonary and activity-regulated chemokine (PARC), periostin, lactate dehydrogenase, eosinophil, total IgE, and antigen-specific IgE are described in US Patent Application Publication No. US2014/0072583 (incorporated by reference in its entirety).

Serum biomarkers that were measured at various time points between screening and Week 32 included: Serum levels of TARC (Human CCL17/TARC Quantikine ELISA kit; R&D Systems, Minneapolis, MN, USA); Serum periostin (Human Periostin/OSF-2 DuoSet 15 Plate; R&D Systems); LDH (Roche Modular and Cobas Analyzers (Roche Diagnostics, Indianapolis, IN, USA); Eosinophil counts determined as part of the differential cell count; and Total IgE and antigen-specific IgEs in serum (measured by ImmunoCap assay (ImmunoCAPR Fluorescent Enzyme Immunoassay; Thermo Scientific, Phadia A B, Uppsala, Sweden).

For allergen-specific IgE, the lower limit of quantitation LLQ) was 0.10 kU/L; a level ≥0.35 kU/L was considered evidence of allergen sensitization.

Mean percent changes in serum biomarkers TARC, periostin and LDH, and median percent changes in total IgE and allergen-specific IgEs were compared at Week 16 for each of the dupilumab dosing regimens versus placebo using an analysis of covariance with the baseline value as a covariate.

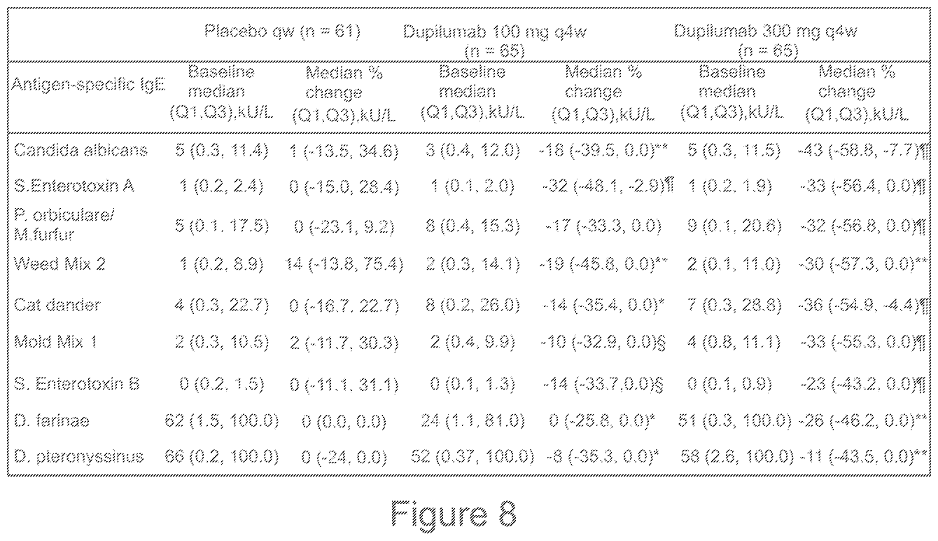

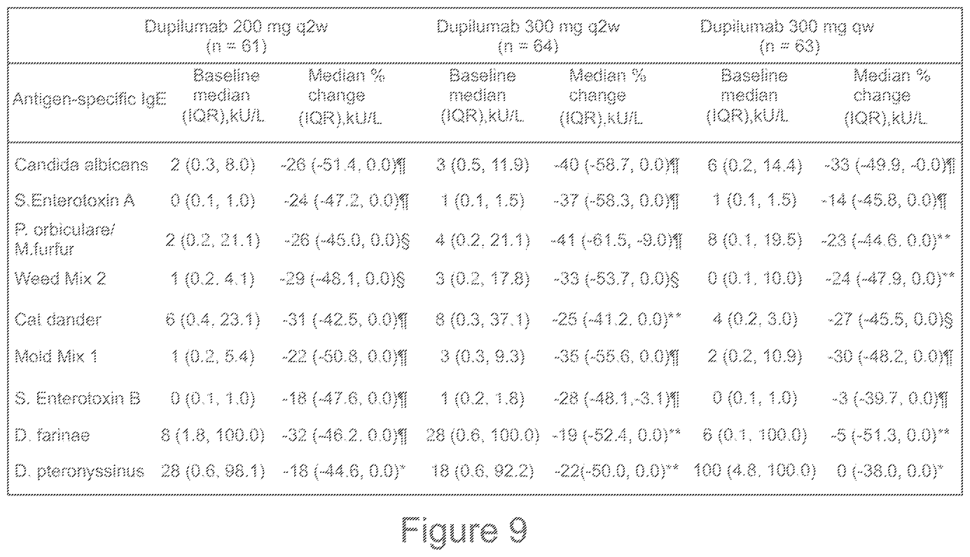

Reductions from baseline to Week 16 with dupilumab vs placebo were observed in multiple antigen-specific IgEs in serum, including those specific for S. aureus enterotoxins (FIGS. 8 and 9).

### Example 3: Staphylococcus aureus Skin Colonization

Skin microbial colonization analysis was conducted on samples taken from subjects who participated in a clinical trial of dupilumab. Subjects with moderate-to-severe atopic dermatitis (AD) were administered 16 weekly doses of either dupilumab (200 mg) or placebo; patients on dupilumab received a loading dose of 400 mg on day 1. S. aureus colonization and infection was determined on AD lesional and non-lesional skin between screening and Week 32. Skin swabs (pre-moistened with Tris-EDTA buffer) were collected from pre-measured skin areas (˜10 cm×10 cm) and tested for the presence of S. aureus. Bacterial cells contained in the swab were lysed, and total genomic DNA purified. The abundance of S. aureus-specific femA DNA from total bacterial genomic DNA was determined using quantitative real-time PCR (qPCR). The relative colony-forming units (rCFU) of S. aureus was determined using a standard curve generated with genomic DNA from known CFU of S. aureus. S. aureus abundance was reported as median (interquartile range [IQR]) percent change from baseline. Data were set to missing after rescue medication. Variables were not adjusted for multiplicity and thus nominal p values are provided.

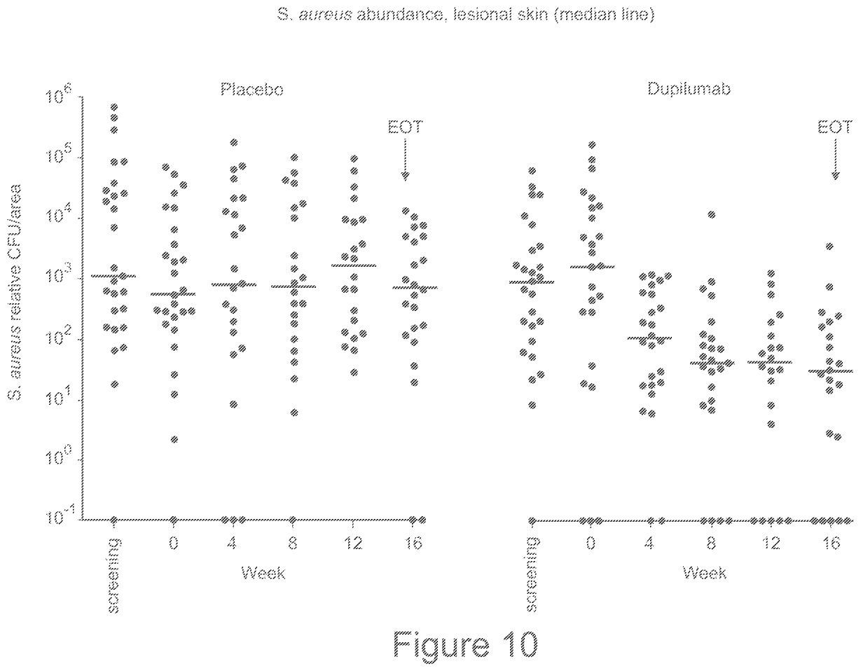

Dupilumab significantly reduced S. aureus abundance in AD lesional skin (median % change from baseline to Week 16 compared with placebo [P=0.0125; Table 3]), and an overall reduction from baseline was observed at Week 16 in median S. aureus abundance compared with placebo (FIG. 10).

TABLE 3
S. aureus abundance in AD lesional and
non-lesional skin after 16-weeks treatment

| | Placebo qw | | Dupilumab | |
|---|---|---|---|---|
| | (n = 27) | | 200 mg qw (n = 27) | |
| | Baseline | Median % | Baseline | Median % |
| | median | change | median | change |
| | (Q1, Q3), | (Q1, Q3), | (Q1, Q3), | (Q1, Q3), |
| | rCFU/area | rCFU/area | rCFU/area | rCFU/area |
| AD lesional | 1289 (239.0, | −49 (−87.7, | 1630 (287.3, | −99 (−99.9, |
| skin | 16149.0) | 635.8) | 15442.5) | −94.1)* |
| AD non- | 166 (101.2, | −11 (−90.7, | 374 (84.8, | −89 (−97.5, |
| lesional skin | 4755.6) | 1063.4) | 3146.4) | −21.6) |

*P < 0.05 vs placebo;
Q1, lower quartile of interquartile range;
Q3, upper quartile of interquartile range;
rCFU, relative colony forming units;
qw, once weekly

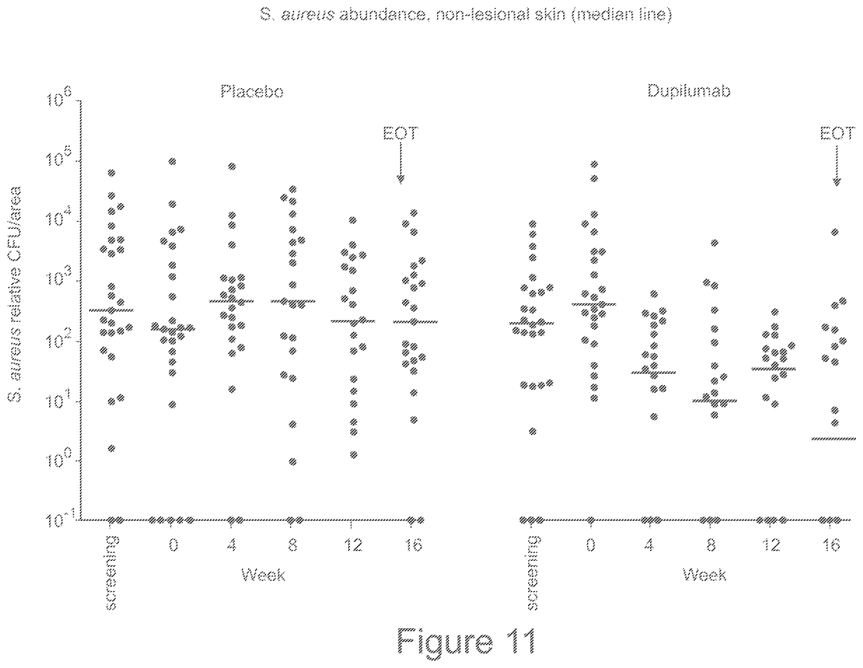

In non-lesional AD skin, the dupilumab group demonstrated a numerically greater reduction in S. aureus abundance compared with placebo (median % change from baseline to Week 16 [Table 3; P=0.9865]), and an overall median reduction from baseline at Week 16 compared with placebo (FIG. 11).

### Example 4: Dupilumab Improves Symptoms of Perennial Allergic Rhinitis (PAR) in Uncontrolled Persistent Asthma Patients with Comorbid PAR

Dupilumab, an anti-interleukin (IL)-4 receptor-α monoclonal antibody, inhibits IL-4 and IL-13 signaling, key drivers of type 2 inflammation. In a pivotal, phase 2b study (NCT01854047), dupilumab improved forced expiratory volume in 1 second, reduced severe asthma exacerbations, improved quality-of-life, and was generally well tolerated in patients with uncontrolled persistent asthma despite use of medium-to-high-dose inhaled corticosteroids plus long-acting R₂-agonists (ICS+LABA). This post-hoc analysis examines the effect of dupilumab on the Sino-Nasal Outcome Test (SNOT-22) total score as well as individual items typically associated with allergic rhinitis (nasal blockage, runny nose, sneezing, and post-nasal discharge) in patients with perennial allergic rhinitis (PAR), a common comorbidity of asthma. PAR was defined by the presence of specific IgE 0.35 Ku/L against perennial antigens (Aspergillus fumigatus, cat dander, D. farinae, D. pteronyssinus, dog dander, German cockroach, or Oriental Cockroach) at study entry. Due to possible confounding effects, patients with comorbid nasal polyposis were excluded from the analysis. Data are reported for the intent-to-treat population who received placebo and either of the dupilumab regimens 200 or 300 mg every 2 weeks [q2w] currently under investigation in phase 3 (NCT02414854). Endpoints were change from baseline to Week 24 in SNOT-22 total score as well as the individual items, post-nasal discharge, nasal blockage, runny nose, and sneezing. Of 392 patients receiving dupilumab (200 or 300 mg q2w) or placebo, 241 (61%) had PAR. In PAR patients, dupilumab 300 mg q2w showed a significant improvement on SNOT-22 total score (LS mean difference −5.98 [95% Cl, −10.45 to −1.51], P=0.009 vs. placebo) and all 4 allergic rhinitis-associated symptoms as defined above relative to placebo (nasal blockage: −0.60 [95% Cl, −0.96 to −0.25]; runny nose: −0.67 [95% Cl, −1.04 to −0.31]; sneezing: −0.55 [95% Cl, −0.89 to −0.21]; and post-nasal discharge: −0.49 [95% Cl, −0.83 to −0.16]; all P<0.01 vs. placebo); dupilumab 200 mg q2w showed a numerical, but not statistically significant decrease in SNOT-22 total score (˜1.82 [95% Cl, −6.46 to 2.83], P=0.443) as well as in the 4 allergic rhinitis-associated symptoms. No differences relative to placebo were observed in non-PAR patients on SNOT-22 total score and the 4 allergic rhinitis-associated symptoms. In conclusion, dupilumab 300 mg q2w significantly improves sinonasal symptoms in patients with uncontrolled persistent asthma and comorbid PAR.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1-8. (canceled)

9. A method for reducing a serum allergen-specific IgE level in a subject comprising administering a therapeutically effective amount of an interleukin-4-receptor (IL-4R) antagonist to the subject in need thereof,
wherein the subject has atopic dermatitis or asthma and has comorbid allergic rhinitis;
wherein the subject has a serum allergen-specific IgE≥0.35 kU/L against an allergen that is an aeroallergen or S. Aureus enterotoxin A or B;
wherein the IL-4R antagonist is an antibody or antigen-binding fragment thereof that binds to IL-4Rα and that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO; 3, the HCDR2 comprises the amino acid sequence of SEQ ID NO; 4, the HCDR3 comprises the amino acid sequence of SEQ ID NO: 5, the LCDR1 comprises the amino acid sequence SEQ ID NO: 6 the LCDR2 comprises the amino acid sequence LGS and the LCDR3 comprises the amino acid sequence of SEQ ID NO: 8.

10. The method of claim 9, wherein the serum allergen-specific IgE level is reduced by at least 20% from baseline following administration.

11. The method of claim 9, wherein the serum allergen-specific IgE level is reduced by at least 5000 from baseline following administration.

12. The method of claim 9, wherein the allergen is selected from the group consisting of Alder Grey, Alternaria Tenuis, Bermuda Grass, Silver Birch, Cat Dander, Cladosporium, Cockroach (German), Dermatophagoides farinae (mite), D. pteronyssinus, Dog Dander, Elm, Johnson Grass, White Oak, Ragweed Short, Mugwort Sage, Timothy (Phleum), White Ash, Candida albicans, Malasezzia furfur, Pityrosporum orbiculare, mold, Staphylococcal enterotoxin A, and Staphylococcal enterotoxin B.

13. The method of claim 12, wherein the IL-4R allergen is Staphylococcal enterotoxin A or Staphylococcal enterotoxin B.

14. The method of claim 9, wherein the IL-4R antagonist is administered at a dose of about 75 mg to about 600 mg.

15. The method of claim 14, wherein the IL-4R antagonist is administered at a dose of about 300 mg.

16. The method of claim 14, wherein the IL-4R antagonist is administered at a dose of about 200 mg.

17. The method of claim 9, wherein the IL-4R antagonist is administered as an initial dose followed by one or more secondary doses, wherein each secondary dose is administered 1 to 4 weeks after the immediately preceding dose.

18. The method of claim 17, wherein the initial dose comprises about 75 mg to about 600 mg of the IL-4R antagonist.

19. The method of claim 17, wherein each secondary dose comprises about 75 mg to about 600 mg of the IL-4R antagonist.

20. The method of claim 17, wherein the initial dose comprises about 600 mg and each secondary dose comprises about 300 mg of the IL-4R antagonist, wherein each secondary dose is administered one week or two weeks after the immediately preceding dose.

21. The method of claim 17, wherein the initial dose comprises about 400 mg and each secondary dose comprises about 200 mg of the IL-4R antagonist, wherein each secondary dose is adminstered one or two weeks after the immediately preceding dose.

22. (canceled)

23. (canceled)

24. The method of claim 9, wherein the subject has moderate-to-severe atopic dermatitis, and comorbid allergic rhinitis.

25. The method of claim 9, wherein the IL-4R antagonist is administered subcutaneously to the subject.

26. The method of claim 9, wherein a second therapeutic agent is administered to the subject before, after, or concurrent with the IL-4R antagonist.

27. The method of claim 26, wherein the second therapeutic agent is selected from the group consisting of an anti-histamine, systemic immunotherapy, a corticosteroid, a long-acting 02-agonist, a tumor necrosis factor (TNF) inhibitor, an interleukin 1 (IL-1) inhibitor, an IL-5 inhibitor, an IL-8 inhibitor, an IgE inhibitor, a non-steroidal anti-inflammatory drug (NSAID), and interferon-gamma (IFNγ).

28-31. (canceled)

32. The method of claim 9, wherein the anti-IL-4Rα antibody or antigen-binding fragment thereof comprises a heavy chain variable region (HCVR) that comprises the amino acid sequence of SEQ ID NO: 1 and a light chain variable region (LCVR) that comprises the amino acid sequence of SEQ ID NO: 2.

33. The method of claim 9, wherein the anti-IL-4Rα antibody comprises a heavy chain comprising the amino acid sequence of SEQ ID NO: 9 and a light chain comprising the amino acid sequence of SEQ ID NO: 10.

34. The method of claim 9, wherein the IL-4R antagonist is dupilumab or a bioequivalent thereof.

35-37. (canceled)
